(19) United States
(12) Patent Application Publication
(10) Pub. No.: US 2025/0389743 A1
(43) Pub. Date: Dec. 25, 2025

(54) METHOD AND INSTRUMENT FOR DRAWING A VOLUME OF A BIOLOGICAL SAMPLE FOR TESTING

(71) Applicant: Beckman Coulter, Inc., Brea, CA (US)

(72) Inventors: Bhagaban Nayak, Bengaluru (IN); Aaron P. O'REILLY, Chaska, MN (US); Kasiviswanathan BOOMINATHAN, Bengaluru (IN); Steven P. RIVERS, Minneapolis, MN (US)

(21) Appl. No.: 18/879,737

(22) PCT Filed: Jun. 8, 2023
(86) PCT No.: PCT/US23/24776

§ 371 Date: Dec. 27, 2024

Related U.S. Application Data
(60) Provisional application No. 63/356,120, filed on Jun. 28, 2022.

(51) Int. Cl. G01N 35/10; A61B 10/00

(52) U.S. Cl. CPC G01N 35/1016; A61B 10/0096

(57) ABSTRACT

A method is provided. The method comprises: accessing test order data, wherein the test order data comprise information specifying a set of clinical tests to be carried out on a biological sample, wherein the set of clinical tests comprises at least a clinical test and the biological sample is contained in a sample container; obtaining test volume data by using the test order data, wherein the test volume data comprise information specifying a test volume; and instructing a pipetting device of a laboratory instrument to draw a drawing volume of the biological sample from the sample container, the drawing volume being based on the test volume data; wherein the test volume is the volume of the biological sample needed to carry out: the clinical test, and one or more supplementary tests associated with the clinical test, whenever one or more test volume conditions associated with the clinical test are met, wherein the one or more test volume conditions comprise a condition that one or more rules require that the one or more supplementary tests shall be carried out whenever one or more supplementary test conditions on the clinical test are met.

### RELATED APPLICATION

This application is related to, and claims the benefit of, provisional patent application 63/356,120, titled “Method and Instrument for Drawing a Volume of a Biological Sample for Testing,” filed in the United States Patent Office on Jun. 28, 2022. That application is hereby incorporated by reference in its entirety.

### TECHNICAL FIELD

The following description relates to the field of biological samples testing.

### BACKGROUND

Analytic and clinical laboratories typically comprise laboratory instruments that are configured to automatically carry out one or more clinical tests (e.g. immunoassay tests) on biological samples (such as blood samples).

Typically, when a tube containing a biological sample is introduced into a laboratory instrument, the instrument reads the barcode on the tube and queries for a test order based on the barcode (either in the connected laboratory information system or in the local instrument console software). Upon receiving the test order, the instrument draws a sample volume based on the volume needed to perform the test(s) in the test order and on an additional volume (hereinafter also referred to as “reserve volume”) for possible rerun(s) of the test(s) and/or for possible reflex test(s) associated to the test(s) in the test order. After the aspiration (also referred to as “drawing”) of the sample volume, the instrument immediately releases the tube, so that it can be used for a different purpose (e.g. moved to another instrument).

In particular, a reflex test associated to a test of a test order is a test that is different from the test of the test order and is carried out if the result of the test of the test order meets one or more reflex criteria. For instance, the reflex criteria may be specified in rules which e.g. may be set by a user of the laboratory instrument.

The reserve volume is a fixed, predetermined volume of the sample, drawn to cope with the possibility of having to rerun the test or run a reflex test. In conventional instruments, however, the aspiration of the reserve volume does not take into account whether the possibility of a rerun or a reflex test is even contemplated or not. Accordingly, sample volume may be wasted by drawing extra volume (the reserve volume) for tests that will not require reruns or reflex tests.

Moreover, typically, the reserve volume is a fixed amount that does not take into account the actual volume needed to rerun the test(s) or to carry out possible reflex test(s). Hence, in some cases, the reserve volume may not be enough for re-running a specific test or for running a specific reflex test, which prompts the instrument to ask the user to introduce additional sample volume in the instrument, leading to a delay in the processing of the test, since the tube needs to be rerouted to the instrument.

### SUMMARY

It is an object of the invention to improve the sample volume aspiration in order to reduce sample waste and to increase the efficiency of sample testing.

The achievement of this object in accordance with the invention is set out in the independent claims. Further developments of the invention are the subject matter of the dependent claims.

According to a first aspect, a method, e.g. a computer-implemented method, is provided. The method comprises:
accessing, e.g. by a first computing device, test order data, wherein the test order data comprise information specifying a set of clinical tests to be carried out on a biological sample, wherein the set of clinical tests comprises at least a clinical test (hereinafter also referred to as: “first clinical test”) and the biological sample is contained in a sample container;
obtaining, e.g. by the first computing device, test volume data by using the test order data, wherein the test volume data comprise information specifying a test volume; and
instructing, e.g. by the first computing device, a pipetting device of a laboratory instrument to draw a drawing volume of the biological sample from the sample container, the drawing volume being based on the test volume data;
   wherein the test volume is the volume of the biological sample needed to carry out:
the clinical test, and
if one or more test volume conditions associated with the clinical test are met, one or more supplementary tests associated with the clinical test,
   wherein the one or more test volume conditions comprise a condition that one or more rules require that the one or more supplementary tests shall be carried out if one or more supplementary test conditions on the clinical test are met.

According to the present invention, a computing device may comprise at least one memory and at least one processor. A computing device may also comprise one or more input/output units. In particular, a computing device may comprise a plurality of processors and/or a plurality of memories. According to the present invention, a computing device, e.g. the first computing device, may be a distributed computing system, e.g. a computing network. For example, the first computing device may comprise a computing device integrated with the laboratory instrument and another computing device remote from the computing device integrated with the laboratory instrument.

In particular, the first computing device accesses test order data, the test order data comprising information specifying a set of clinical tests to be performed on a biological sample according to one or more rules, wherein the set of clinical tests comprises at least a clinical test and the biological sample is contained in a sample container.

The biological sample (or simply “sample”) may be a sample of a bodily fluid of a human or animal subject. For example, the bodily fluid may be a physiological fluid, such as blood, saliva, urine, sweat, amniotic fluid, cerebrospinal fluid, ascites fluid, or the like. The biological sample may be put into a sample container after collection and it may be held in the container while being processed. The sample container may be a sample tube. Typically, sample tubes comprise a closed tube end and an end opposite thereto. The latter end defines an opening for inserting the sample in the sample tube. The opening may be closed, e.g. sealed, by a cap.

A clinical test may comprise one or more procedures that, when carried out on the biological sample, allow for estimating the value of a parameter, e.g. a clinical parameter. In particular, a clinical test may comprise physical, biological, optical, mechanical, immunological, and/or chemical procedures. Exemplarily, a clinical test may be an immunoassay test.

Generally, a set may comprise one or more elements. Accordingly, the set of clinical tests comprises at least one clinical test, i.e. one or more clinical tests. In the following, the method will be described with reference to one clinical test, however it is to be understood that, if the set of clinical tests comprises a plurality of clinical tests, the same applies for each clinical test of the set of clinical tests.

The test order data may comprise a set of identifiers. In particular, each identifier of the set of identifiers uniquely identifies a respective clinical test of the set of the clinical tests, wherein said each identifier contains information specifying said respective clinical test. The identifiers of the set of identifiers may be alphanumeric strings. For instance, the alphanumeric string “TSH” uniquely identifies the thyroid-stimulating hormone test and the string “Lp-PLA2” uniquely identifies the Lipoprotein-Associated Phospholipase A2 test.

In the present disclosure, “accessing data” may comprise retrieving the data e.g. from the at least one memory of the first computing device, from the memory of another computing device, or from another remote data storage (a database, a secondary memory, a cloud storage or the like). Accordingly, in some cases, retrieving data may comprise downloading data.

Additionally or alternatively, “accessing data” may comprise receiving the data, e.g. from a user or a computing device different from the first computing device. The two options are not mutually exclusive. For instance, accessing, data may comprise receiving the data, storing the data in the memory of the first computer device and retrieving the data by accessing said memory.

Exemplarily, the sample container may comprise an indicium, e.g. a barcode, which may for example be securely affixed, e.g. by means of adhesive, to the sample container, for instance to its wall or its cap. The test order data may be retrieved, e.g. by the first computing device, by reading indicium data from the indicium on the sample container. To this aim, a data reading unit may be comprised in the first computing device or functionally connected to the first computing device. The data reading unit may comprise an optical scanner configured to decode a barcode or a QR code. In another example, the indicium may be an RFID tag and the data reading unit may comprise an RFID reader.

In one example, the test order data may be fully comprised in or coincide with the indicium data. In this case, the test order data are “directly” read from the sample container, meaning that the information content of the test order data is fully present on the sample container, more precisely in the indicium.

In another example, the test order data may be “indirectly” read from the sample container, meaning that the sample container only acts as an intermediary between the source of the test order data (e.g. a remote database) and the first computing device. In other words, the data reading unit may obtain from the indicium a link (the indicium data) for retrieving the first data from a source. For instance, a QR code on the sample tube may be scanned by the data reading unit to obtain such a link. In this case, the test order data and the indicium data are completely distinct.

In yet another example, a portion of the test order data may be directly read and another portion may be indirectly read. In this case, the test order data may partially overlap with the indicium data. For instance, the indicium data may comprise the identifier identifying the clinical test, wherein the identifier is also part of the test order data. By using the identifier stored in the indicium, the first computing device may retrieve the rest of the test order data from another source, e.g. its own memory or a remote data storage.

In particular, the laboratory instrument may be configured to process the sample according to one or more rules (hereinafter also referred to as: “instrument rules”). In particular, a rule specifies one or more actions that the laboratory instrument is required to carry out, if one or more conditions are met.

For example, a rule may require that a supplementary test shall be carried out by the laboratory instrument if a respective supplementary test condition is met. In other words, the supplementary test is carried out if the supplementary test condition is met, while the supplementary test is not carried out if the supplementary test condition is not met. In some cases, a rule may require that a plurality of supplementary tests shall be carried out if one supplementary test condition is met. In other cases, a rule may require that a plurality of supplementary tests shall be carried out if a plurality of respective supplementary test conditions are met.

For instance, a rule refers to a respective clinical test of the clinical tests that the laboratory instrument is configured to carry out. If this is the case, the rule and the respective clinical test are associated with one another. Exemplary, a rule may comprise (i) information specifying the respective clinical test associated thereto, (ii) the one or more supplementary tests, and (iii) the one or more supplementary tests conditions that, if met, prompt the laboratory instrument to carry out the one or more supplementary tests.

A clinical test of the set of clinical tests may have zero, one or a plurality of rules associated to it. For instance, at least one clinical test of the clinical tests that the laboratory instrument is configured to carry out may not refer to any rules of the one or more rules. For example, two different rules may be associated to the same clinical test of the clinical tests that the laboratory instrument is configured to carry out. Hence, more particularly, some clinical tests of the set of clinical tests may be associated with at least a respective rule of the one or more rules, while some other clinical tests of the set of clinical tests are not.

In general, the laboratory instrument may be configured to carry out an instrument-specific set of clinical tests, e.g. according to the one or more rules. The instrument-specific set of clinical tests is a set consisting of each and every clinical test that the instrument is capable of carrying out. In particular, the set of clinical tests according to the first aspect of the present invention is a subset, e.g. a proper subset, of the instrument-specific set of clinical tests. Indeed, the set of clinical tests comprises those tests ordered for a specific sample, while the instrument-specific set of clinical tests comprises all tests that the instrument can perform (e.g. depending on its configuration/design).

More particularly, the laboratory instrument is configured, when carrying out clinical tests, to abide by each rule of the one or more rules, i.e. to carry out, if the one or more conditions associated with said rule are met, the one or more actions required by said rule. For instance, the laboratory instrument is configured, when carrying out a clinical test of the instrument-specific set of clinical tests, (i) to assess the presence of one or more rules associated to said test, (ii) to check whether the one or more supplementary tests conditions are met and (iii) if the one or more supplementary tests conditions are met, to carry out the one or more supplementary tests.

Since the one or more supplementary test conditions are conditions on the clinical test with which the rule is associated, it may be said that the one or more supplementary tests are associated with the clinical test. The latter may also be referred to as “primary test”. Accordingly, the set of clinical tests may comprise one or more primary tests. In particular, the rule may require that the one or more supplementary test are carried out by the laboratory instrument after the primary test to which it is associated has been carried out.

A supplementary test is also a clinical test, however its execution is contingent on the supplementary test condition(s) being met, while the primary test is always to be performed. In some examples, the execution of one supplementary test may be contingent on one supplementary test condition, while in other examples the execution may be contingent on a plurality of supplementary test conditions. One or more supplementary tests may be associated with the clinical test.

As explained, the one or more supplementary test conditions are conditions that, when met, triggers the execution of one or more supplementary test. The one or more supplementary test conditions are met if the clinical test satisfies certain criteria. In one example, the one or more supplementary test conditions may consist of the condition that the clinical test is a predetermined clinical test. In this case, every time the primary test is run, the supplementary test is also run. In another example, the one or more supplementary test condition may consist of a result condition on a result of the clinical test. In other words, if the result of the clinical test meets the result condition, the supplementary test is performed. Accordingly, a rule may require that a supplementary test shall be carried out by the laboratory instrument if a result condition on the result of the clinical test is met.

The execution of a clinical test may lead to one or more results, wherein each result may be a numerical value or an alphanumeric string. The result condition may be a match condition, according to which the result has to be identical to a predetermined value or string. For instance, the result of a primary clinical test may be either the string “acceptable” or the string “unacceptable”. If this is the case, the match condition may be the condition that the result of the primary test is equal to “unacceptable”. Alternatively, the result condition may be a threshold condition, according to which the result has to be less than or greater than a predetermined value, or the result has to be within a predetermined range, i.e. between two predetermined values.

Exemplarily, the supplementary test may be a rerun test, i.e. the supplementary test may coincide with the primary test. If this is the case, the primary test may be performed twice, e.g. if a result indicates an anomaly (e.g. the result is outside a predetermined range). Accordingly, if the clinical test meets the one or more supplementary test condition, the rerun test is performed, that is, the clinical test is repeated.

Exemplarily, the supplementary test may be a reflex test, i.e. the supplementary test may be different from the primary test, and it may be a clinical test carried out e.g. to confirm a result of the primary test by different means or to disambiguate a result of the primary test.

In some examples, a rule of the one or more rules may require to carry out the same reflex test a plurality of times. In this case the reflex test may also be referred to as: “replicate reflex test”. Accordingly, a rule may specify that a given supplementary test may be carried out more than once.

In particular, the one or more rules may be generated by a user of the laboratory instrument, e.g. to implement the workflow of the laboratory comprising the laboratory instrument. The one or more rules may be generated by the user with the aim of improving the reliability of the test results. For example, the one or more rules may require to run a secondary test if the result of the first test falls within a range of values that renders this result inconclusive and/or, according to some reliability requirements, not reliable enough.

The test volume data may comprise a numerical value indicating a volume value, e.g. expressed in cubic centimetres, millilitres, or microliters Said volume value is in particular the test volume. The test volume always accounts for the volume of biological sample needed to carry out the first clinical test (which may be referred to as “primary volume”). In other words, the test volume comprises at least the primary volume.

The test volume may also account for the volume of biological sample needed to carry out one or more supplementary tests. In particular, this is the case if one or more test volume conditions associated with the first clinical test are met. The volume needed to carry out a given supplementary test may be referred to as “supplementary volume”. Accordingly, the test volume comprises one or more supplementary volumes (besides the primary volume), if one or more test volume conditions are met.

The one or more test volume conditions comprise at least a condition that the one or more rules require that the one or more supplementary tests shall be carried out if their respective supplementary test conditions are met. Herein, the latter condition may also be referred to as: “rule condition”. In particular, the rule condition is the existence of at least one rule of the one or more rules that: (i) is associated with the first clinical test and (ii) introduces the possibility of having to run supplementary test(s). Indeed, as mentioned above, the first clinical test may or may not be associated with a rule encompassing potential supplementary test(s).

Accordingly, the method may also be considered as comprising:
accessing, e.g. by the first computing device, test order data, wherein the test order data comprise information specifying a set of clinical tests to be carried out on a biological sample, wherein the set of clinical tests comprises at least a clinical test (also referred to: “first clinical test”) and the biological sample is contained in a sample container;
obtaining, e.g. by the first computing device by using the test order data, test volume data, wherein the test volume data comprise information specifying a test volume;
instructing, e.g. by the first computing device, a pipetting device of a laboratory instrument to draw a drawing volume of the biological sample from the sample container, the drawing volume being based on the test volume data;
   wherein the test volume is the volume of the biological sample needed to carry out:
the clinical test, and
whenever one or more test volume conditions associated with the clinical test are met, one or more supplementary tests associated with the clinical test,
   wherein the one or more test volume conditions comprise a condition that at least one rule is defined for the clinical test, wherein the rule requires that the one or more supplementary tests shall be carried out whenever one or more supplementary test conditions on the clinical test are met.

As mentioned above, the test volume may include extra volume besides the one needed to run the first clinical test only if there is actually a possibility that further tests may be run. In particular, in such cases, the extra volume corresponds to the volume actually needed to run the one or more supplementary tests associated with the first clinical test.

In the present disclosure, “obtaining data” may comprise accessing the data as discussed above. Alternatively, “obtaining data” may comprise generating the data, e.g. creating the data based on one or more inputs. In yet another example, “obtaining data” may comprise accessing a first portion of the data and generating a second portion of the data e.g. from the first portion of the data.

According to the first aspect of the present invention, the pipetting device of the laboratory instrument is instructed, e.g. by the first computing device, to draw the drawing volume. In particular, the pipetting device of the laboratory instrument is instructed, e.g. by the first computing device, to draw, before the first clinical test is carried out, the drawing volume of the biological sample from the sample container.

The laboratory instrument may be configured to carry out the instrument-specific set of clinical tests and may, thus, be an analytic instrument. In particular, an analytic instrument (e.g. analyser) is an instrument configured to carry out one or more analytic steps, such as measuring one or more characteristics of the sample, e.g. the concentration of a given analyte.

The laboratory instrument comprises the pipetting device, which is configured to draw a portion of the biological sample from the sample container. In other words, the pipetting device is capable of coming into contact with the biological sample contained in the sample container (e.g. by moving a robotic arm and entering at least partially the sample container) and aspiring a portion of the biological sample (e.g. by means of a pipette).

Exemplarily, the first computing device may be comprised in the laboratory instrument and may be configured to control the operations of the laboratory instrument, and, in particular, of its pipetting device. Alternatively, the laboratory instrument may comprise another computing device (a second computing device). In this case, the first computing device may be remote from the laboratory instrument and configured to communicate with the second computing device, which is configured to control the laboratory instrument. In yet another example, as described above, the first computing device may be a distributed computing system comprising a computing device integrated with the laboratory instrument and another computing device remote from the computing device integrated with the laboratory instrument.

The drawing volume is based on the test volume data in that it coincides with the test volume or it is computed by using the test volume data. For example, if the set of clinical tests comprises only one clinical test, i.e. the first clinical test, the drawing volume may coincide with the test volume. Alternatively, if the set of clinical tests comprises a plurality of clinical tests, the drawing volume may be computed by adding the test volumes of all the clinical tests.

In particular, according to the present invention, there are two possible scenarios that can unfold when a clinical test is to be performed on a biological sample. In a first scenario, whenever if the first clinical test (in particular a result thereof) satisfies one or more supplementary test conditions, one or more supplementary tests will also have to be carried out, while, whenever the first clinical test does not satisfy any supplementary test condition, no supplementary test will be carried out. In the second scenario, the first clinical test is carried out and it does not trigger in any case the execution of one or more supplementary tests.

According to the method described above, in a case in which there is no possibility of having to carry out any supplementary test, the drawing volume provided to the laboratory instrument for aspiration corresponds to the amount of biological sample needed by the laboratory instrument for carrying out the first clinical test. This way, the amount of unnecessary volume drawn from the biological sample is reduced, thereby reducing the amount of biological sample wasted, i.e. not used to carry out analytical tests.

Conversely, in a case in which there is a possibility of having to carry out one or more supplementary tests, the drawing volume provided to the laboratory instrument for aspiration corresponds to the amount of biological sample needed by the laboratory instrument for carrying out the first clinical test and the one or more supplementary tests. Hence, if the supplementary tests must indeed be carried out, the laboratory instrument has already the quantity of biological sample that it needs. Accordingly, there is no delay in processing the sample, which may be otherwise caused e.g. by having to reroute the sample container to the laboratory instrument.

Therefore, the method leads to a reduction of sample waste and to an increase in the efficiency of sample testing.

Exemplarily, the first clinical test may be associated with an identifier (hereinafter also referred to as: “first identifier”) identifying the first clinical test. If this is the case, obtaining the test volume data may comprise querying a database using the first identifier.

More generally, if the laboratory instrument is configured to carry out an instrument-specific set of clinical tests, e.g. K clinical tests, each identifier of K distinct identifiers is associated with one respective clinical test of the instrument-specific set of clinical tests.

In a first example, the database may a relational table which associates each identifier of the K distinct identifiers with one respective volume value, said volume being the volume needed to carry out the clinical test associated with said each identifier and, if contemplated by the one or more rules, to carry out the supplementary tests associated with the clinical test associated with said each identifier.

In this example, obtaining the test volume data may comprise accessing the test volume data, which consist of the volume value associated with the first identifier. In this instance, the differentiation between a case in which the execution of the clinical test may lead to the execution of supplementary tests and a case in which the execution of the clinical test will never lead to the execution of supplementary tests is implicitly contained in the volume value. In other words, the differentiation was made prior to storing the volume value in the database, e.g. when determining the volume value.

In a second example, the database may associate each identifier of the K distinct identifiers with one respective first volume value and, if the test associated with said each identifier is associated with one or more supplementary tests, at least a respective second volume value. In particular, the respective first volume value is the volume needed to carry out the clinical test associated with said each identifier and, when present, the respective second volume value is the volume needed to carry out supplementary tests associated with the clinical test associated with said each identifier.

In this example, obtaining the test volume data may comprise accessing the test volume data, which consist of the first volume value associated with the first identifier and, whenever the test volume conditions are met, the second volume value associated with the first identifier. Additionally, whenever the test volume conditions are met, obtaining the test volume data may comprise generating the test volume data by adding the first volume value associated with the first identifier and the second volume value associated with the first identifier.

In an example, the method according to the present invention may comprise determining, by using the rule data, whether the one or more test volume conditions are met. According to the present invention, for instance, the step of determining whether one or more test volume conditions are met may be carried by the first computing device.

Exemplarily, obtaining the test volume data may comprise accessing, e.g. by the first computing device, rule data, the rule data comprising the one or more rules; and determining, by using the rule data, whether one or more test volume conditions are met.

The test order data may comprise the rule data, in which case the step of accessing the rule data may be comprised in the step of accessing the test order data. Alternatively, the rule data may be separate from the test order data. Exemplarily, the rule data may be accessed by using the test order data, e.g. by retrieving the rule data associated with the first clinical test.

The rule data may comprise statements including strings and variables for defining the rules, e.g. in a specific data format or following a specific syntax, such as the syntax of a programming language. Determining whether the one or more test volume conditions are met may comprise parsing the rule data to identify whether at least a rule in the rule data (i) refers to the first clinical test and (ii) requires that, whenever one or more supplementary test conditions are met, one or more supplementary tests shall be carried out.

Accordingly, in the third example, it is checked whether, according to the one or more rules, the first clinical test will never trigger the execution of supplementary tests or whether there is a possibility that one or more supplementary tests will be executed. In the latter case and if the one or more test volume conditions are met, the method may further comprise determining the one or more supplementary tests associated with the clinical test. For instance, the computing device may determine the one or more supplementary tests by using the rule data, e.g. by parsing the rule data.

Checking, e.g. by the first computing device, whether the one or more test volume conditions are met, instead of retrieving fixed values from a database leads to a greater flexibility. Indeed, different rules may be associated with the first clinical test, e.g. by different users and/or within different contexts. In this example, the test volume is dynamically computed every time the first clinical test is run and it is not hard-coded.

Exemplarily, the method according to the present invention may comprise accessing, e.g. by the first computing device, the rule data; and generating, e.g. by the first computing device, a database by using the rule data. In particular, this database may be the database of the first and the second examples described above. The step of creating the database may comprise determining, by using the rule data, whether the one or more test volume conditions are met.

For example, if the database is the database of the second example described above, determining whether the one or more test volume conditions are met allows for determining the volume value associated to the first identifier. In particular, if the database is the database of the second example described above, determining whether the one or more test volume conditions are met allows for determining whether the database should store a second volume value associated with the first identifier or not and, in the affirmative case, for determining the value of said second volume.

In some examples (e.g. in the second example and in the third example), obtaining the test volume data may further comprise:
obtaining a primary value indicating a volume needed to carry out the first clinical test and at least a secondary value indicating a volume needed to carry out the one or more supplementary tests associated with the first clinical test; and
computing the test volume by using the primary value and the at least secondary value.

In some examples, e.g. in the second example, the primary value is the first value associated with the first identifier and the secondary value is the second value associated with the first identifier.

In particular, the step of obtaining the primary value and the at least secondary value may be carried out if the one or more test volume conditions are met. Consequently, also the step of computing the test volume may be carried out if the one or more test volume conditions are met.

As already mentioned above, when the set of clinical tests comprises a plurality of clinical tests, the steps described herein may be performed for each clinical test of the set of clinical tests. In particular, the method may be considered as comprising:
accessing, by a computing device, test order data, wherein the test order data comprise information specifying a set of clinical tests to be carried out on a biological sample according to one or more rules, wherein the set of clinical tests comprises a plurality of clinical tests and the biological sample is contained in a sample container;
obtaining, by the computing device by using the test order data, test volume data, wherein the test volume data comprise information specifying a set of test volumes, each test volume of the set of test volumes corresponding to a respective clinical test of the set of clinical tests;
obtaining, by the computing device by using the test volume data, a drawing volume; and
instructing, by the computing device, a pipetting device of a laboratory instrument to draw the drawing volume of the biological sample from the sample container;
   wherein, for each clinical test of the set of clinical tests, the respective test volume is the volume of the biological sample needed to carry out:
said each clinical test, and
whenever one or more test volume conditions associated with said each clinical test are met, one or more respective supplementary tests associated with said each clinical test,
   wherein the one or more test volume conditions associated with said each clinical test comprise a condition that the one or more rules require that the one or more respective supplementary tests shall be carried out whenever one or more respective supplementary test conditions on said each clinical test are met.

In particular, the drawing volume may be equal to the sum of the test volumes specified in the test volume data. More particularly, the set of clinical test consists of N clinical tests, C₁, C₂, . . . C_{N}, and the test volume associated with the clinical test Cⱼ (j=1, 2, . . . , N) is indicated by Vⱼ. In this case, for instance, the drawing volume Va reads as follows:

$${V}_{d} = {\sum }_{j = 1}^{N} {V}_{j} .$$

According to an example, the one or more supplementary test conditions may comprise at least a result condition. According to one example, the method may further comprise:
obtaining, e.g. by the first computing device, probability data, wherein the probability data comprise information specifying a probability that a result of the first clinical test meets the result condition; and
determining, e.g. by the first computing device by using the probability data, whether the probability that the result of the first clinical test satisfies the result condition exceeds a probability threshold;
   wherein the one or more test volume conditions further comprise the condition—hereinafter also referred to as: “probability condition”—that the probability that the result of the first clinical test satisfies the result condition exceeds the probability threshold.

In particular, the step of obtaining the probability data and the step of determining whether the probability that the result of the first clinical test satisfies the result condition exceeds a probability threshold may be carried out if the one or more rules require that the one or more supplementary tests shall be carried out if the one or more supplementary test conditions on the first clinical test are met.

The probability data may comprise a probability value, expressed e.g. as a percentage or as a real number between 0 and 1, that indicates the probability that a result of the first clinical test meets the result condition. In particular, the probability value quantifies the likelihood that the one or more supplementary tests will actually be carried out.

In particular, determining whether the probability exceeds a probability threshold is equivalent to checking whether the probability condition is met.

The probability threshold is a numerical value which, like the probability, may be expressed as a percentage or as a real number between 0 and 1. The probability threshold may be a fixed value, e.g. a hardcoded value, that is always the same for the related result condition(s).

In this example, the one or more test volume conditions comprise at least two conditions, namely the rule condition and the probability condition. Besides the mere possibility of the supplementary test(s) having to be carried out, the likelihood of it happening is also factored in. Thus, the approach is more fine-tuned and, in particular, it may reduce sample waste by avoiding that the pipetting device draws extra volume when the extra volume will only be needed in very few cases. More particularly, taking into account the probability allows for weighing the risk of sample waste against the efficiency of testing and determine a test volume that optimises both.

In some examples, obtaining the probability data may comprise: accessing history data, the history data comprising information specifying how often the result condition is met; and generating the probability data using the history data.

The probability data may be data derived e.g. from medical literature and/or from history data, wherein the history data comprise information specifying how often the result condition is met by the result of the first clinical test, namely how often the result of the first clinical test has satisfied the result condition in past executions of the clinical test. Thus, the history data may comprise a record of all the times the first clinical test was carried out and a record of the results of the first clinical test in all these instances. Additionally or alternatively to the record of the results, the history data may comprise statistics about the results in relation to the result condition, e.g. a flag for each execution of the first clinical test indicating whether the result in that occurrence met the result condition.

Generally, the history data may be data collected from previous executions of the clinical test and stored e.g. in a database, such as a local database or a cloud database. In some examples, the history data may relate to a single instrument, namely the laboratory instrument to which the pipetting device belongs. In other examples, the history data may be collected from a plurality of laboratory instruments capable of carrying out the clinical test. The plurality of laboratory instruments may include instruments within the same laboratory or instruments across different laboratories, e.g. within the same geographical region as defined by national or other administrative borders. The instruments are instruments of the same type that are configured to perform the first clinical test.

For instance, the first computing device may determine a number indicating how many times the result condition was met, e.g. by looking at the results themselves or by looking at the flags, and compute the probability value by dividing this number by the total number of times the clinical test was carried out.

In other examples, the probability data may have been previously derived from the medical literature and/or from history data and the first computing device may simply access the probability data.

After obtaining the probability data, the first computing device may determine, by using the probability data, whether the probability that the result of the first clinical test satisfies the result condition exceeds a probability threshold. In other words, the probability is evaluated against the probability threshold and it is assessed whether the probability is greater than the probability threshold. Thus, a comparison between two numerical values is carried out.

The use of history data for generating the probability data makes the estimation of the probability more accurate. In particular, it can be more accurately determined in which cases it is unlikely that the supplementary test(s) would be carried out, and, thus, drawing a reserve volume is more likely to lead to a waste of sample than not. Accordingly, a greater waste reduction is achieved.

For example, the probability threshold may be dynamically selected, e.g. based on circumstances of the clinical test. In some examples, the probability threshold may be selected among a plurality of predetermined probability thresholds based on one or more selection criteria, the one or more selection criteria comprising information specifying any or any combination of: an instrument configured to carry out the clinical test, a laboratory configured to carry out the clinical test, an amount of volume of biological sample contained in the sample container, a category of a subject to whom the biological sample belongs, a priority of the biological sample.

In particular, the method according to the first aspect of the present invention comprises the step of selecting, e.g. by the first computing device, the probability threshold among a plurality of predetermined probability thresholds based on one or more selection criteria.

In particular, each predetermined probability threshold may be associated with one or more selection criteria, in that, whenever the one or more selection criteria apply to the specific conditions under which the first clinical test is carried out by the laboratory instrument, the probability threshold will be selected to be said predetermined probability threshold. Accordingly, the probability threshold may take on different values depending on which criteria apply to the specific case.

A selection criterium may comprise information about an instrument on which the clinical test will be carried out, such as a model or a manufacturer of the instrument. For instance, when the model is an older model that takes a longer time to receive/release the sample container and draw a volume of biological sample from it, when compared to a newer model, the probability threshold for the older model may be lower than the probability threshold for the newer model, for the sake of processing efficiency.

Another selection criterium may comprise information about a laboratory in which the clinical test will be carried out, e.g. a size of the laboratory, which may be expressed by means of various quantities, such as physical size, number of instruments, throughput. For instance, in a laboratory with a bigger size, the transport of sample containers may take considerably more time than in a laboratory of a smaller size, so that rerouting a sample container may be rather undesirable. Accordingly, the probability threshold for the bigger laboratory may be lower than the probability threshold for the smaller laboratory, for the sake of processing efficiency.

Another selection criterium may comprise information about the amount of volume of biological sample contained in the sample container. A probability threshold associated with a smaller available volume may be greater than the probability threshold associated with a larger available volume, so as to avoid wasting precious biological material.

The same may apply for a selection criterium concerning the category of a subject from whom the biological sample was taken. A probability threshold associated with a more vulnerable subject may be greater than the probability threshold associated with a less vulnerable subject, so as to avoid wasting precious biological material. For instance, in the case of a paediatric subject, the probability threshold may be about 1 or 100%.

Conversely, a sample with priority (such as a STAT sample) may have a lower probability threshold with respect to a sample without priority, such as about 0 or 0%.

The plurality of probability thresholds with their associated criteria may be stored in a database. The first computing device may determine whether a selection criterium applies by accessing biological sample data, wherein the biological sample data may comprise information specifying features of the biological sample (e.g. priority and/or volume of the sample and/or subject category), and/or equipment data comprising information about the instrument and the laboratory. Exemplarily, the biological sample data and the test order data may form a single data set.

By having a probability threshold that changes depending on the circumstances of the clinical test, the determination of the test volume may be adapted case by case.

In case of a plurality of supplementary tests, the probability data may comprise a plurality of probability values, each probability value indicating the probability that the result of the first clinical test meets a respective result condition. For example, this may be the case whenever the first clinical test is associated to a plurality of rules, each rule of the plurality of rules specifying a respective result condition.

In particular, according to an example, the one or more supplementary tests consist of a plurality of supplementary tests. If the one or more rules require that the plurality of supplementary tests shall be carried out if the one or more supplementary test conditions on the first clinical test are met, the method may further comprise:
obtaining, e.g. by the first computing device, probability data, wherein the probability data comprise information specifying, for each supplementary test of the plurality of supplementary tests, a respective probability that a result of the first clinical test meets the respective result condition; and
determining, e.g. by the first computing device by using the probability data and for each supplementary test of the plurality of supplementary tests, whether the probability that the result of the first clinical test satisfies the respective result condition exceeds a respective probability threshold.

In this example, the one or more test volume conditions further comprise the condition that, for each supplementary test of the plurality of supplementary tests, the probability that the result of the first clinical test satisfies the respective result condition exceeds a respective probability threshold.

There may be a single threshold applicable to all probabilities, e.g. all the respective probability thresholds are equal with one another, or they may be a plurality of thresholds, each threshold applicable to a respective probability.

In an illustrative example, four supplementary tests {T1, T2, T3, T4} may be associated with the first clinical test, wherein the supplementary tests have respective supplementary volumes {V1, V2, V3, V4} and respective probabilities of a respective result condition being met of {0.9, 0.8, 0.4, 0.3}. The (single) threshold probability may be 0.5, so that only the probability conditions relative to the result conditions of T1 and T2 are met. Accordingly, the test volume, VT, would be given by the sum of the primary volume, VP, for the first clinical test, of V1 and of V2, i.e. VT=VP+V1+V2

Besides the rule condition and if present, the probability condition, the one or more volume test conditions may comprise additional conditions. In an example, the volume test conditions may comprise a condition on an amount of volume of biological sample contained in the sample container and/or a condition on a category of a subject to whom the biological sample belongs.

The condition on an amount of volume of biological sample contained in the sample container may require that this amount of volume be greater than a volume threshold. Thus, in this case, the supplementary volume(s) may not be drawn if the available amount of volume is too small, e.g. to avoid that a further processing of the sample be prevented by taking all its volume.

The condition on a category of a subject to whom the biological sample belongs may require that the subject be not in a vulnerable category, such as geriatric, paediatric or chronically/terminally ill. Accordingly, it is ensured that no amount of the biological sample is wasted, to try and spare a vulnerable subject additional sample taking procedures.

As discussed above, in some examples, obtaining the test volume data comprises determining whether the one or more test volume conditions are met. In case of a plurality of test volume conditions, the different conditions may be checked sequentially, one after the other, e.g. according to a hierarchy. The order in the hierarchy may be determined by the computational effort needed to check a given conditions, wherein the most computationally intensive conditions are at the bottom of the hierarchy, i.e. checked for last only if needed, e.g. whenever the previously checked conditions are met. Additionally or alternatively, a second condition may be contingent on a first condition being met, so that the second condition is lower in the hierarchy. For instance, the probability condition is contingent on the rule condition.

In one example, the computing may determine whether the one or more test volume conditions are met by checking the conditions in the following order: (i) (if present) the condition on the category of the subject; (ii) (if present) the condition on the amount of volume of biological sample contained in the sample container; (iii) the rule condition; and (iv) (if present) the probability condition.

In a further example, the computing may determine whether the one or more test volume conditions are met by checking the conditions in the following order: (i) the rule condition; (ii) (if present) the condition on the category of the subject; (iii) (if present) the condition on the amount of volume of biological sample contained in the sample container; and (iv) (if present) the probability condition.

In a particular example, the method may further comprise drawing, by the pipetting device, the drawing volume of the biological sample from the sample container.

In an example, the method according to the first aspect of the present invention may comprise the step of obtaining, e.g. by the first computing device by using the test volume data, the drawing volume. In this example, the drawing volume may be obtained by adding the test volume to a further volume. In particular, the further volume is the volume needed to carry out other tests of the instrument-specific set of tests, which are not included in the set of clinical tests. For instance, the further volume is the volume needed to carry out each test of these other tests and, when said each test is associated with one or more respective supplementary tests, said respective supplementary tests.

A second aspect of the present invention relates to a computing device comprising a processor configured to perform the method described heretofore. The first computing device is in particular a computing device according to the second aspect of the present invention.

Yet another aspect of the present invention relates to an analytic instrument configured to perform one or more clinical tests on a biological sample. The at least one analytic instrument comprises the computing device according to the previous aspect. and a pipetting device. The computing device is configured to instruct the pipetting device to draw the drawing volume of a biological sample from a sample container and the pipetting device is configured to draw the drawing volume of the biological sample from the sample container.

According to another aspect of the invention, a computer program product is provided. The computer program product comprises instructions which, when the program is executed by a computer, cause the computer to carry out the method described herein. In particular, the computer may be the computing device according to the second aspect of the present invention.

A further aspect of the present invention refers to a computer-readable medium, e.g. a transitory computer readable medium comprising instructions which, when executed by a computer, cause the computer to carry out the method described herein. In particular, the computer may be the computing device.

### BRIEF DESCRIPTION OF THE DRAWINGS

Details of exemplary embodiments are set forth below with reference to the exemplary drawings. Other features will be apparent from the description, the drawings, and from the claims. It should be understood, however, that even though embodiments are separately described, single features of different embodiments may be combined to further embodiments.

FIG. 1 shows a schematic representation of an analytic instrument and of the first computing device.

FIG. 2 shows a flow chart of a method for determining a volume to be drawn.

FIGS. 3a to 3c show flow charts of methods for obtaining a test volume.

FIGS. 4a to 4d show examples of rules.

FIG. 5 shows a flow chart of a method for determining a volume to be drawn.

FIG. 6 shows a flow chart of a method for obtaining a test volume.

### DETAILED DESCRIPTION

In the following, a detailed description of examples will be given with reference to the drawings. It should be understood that various modifications to the examples may be made. Unless explicitly indicated otherwise, elements of one example may be combined and used in other examples to form new examples.

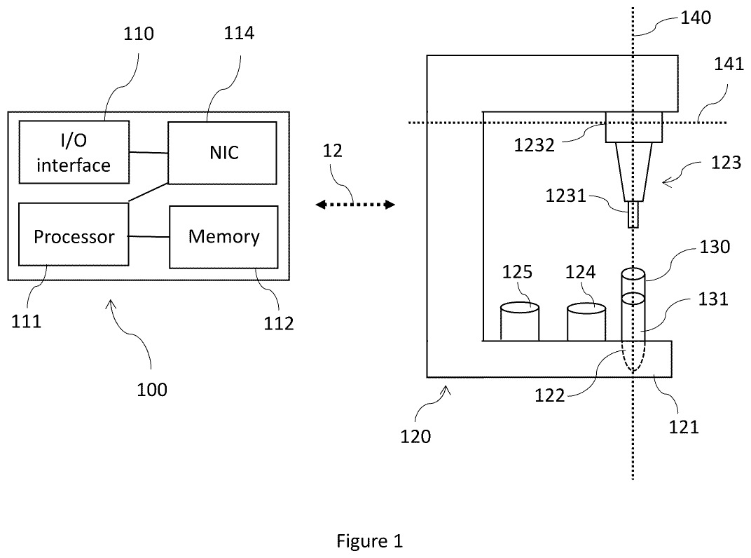

FIG. 1 shows a schematic representation of an analytic instrument 120 and the first computing device 100. In one example, the analytic instrument 120 comprises the first computing device 100. In another example, the first computing device 100 is separate from the analytic instrument 120. In any case, the first computing device 100 is configured to control the analytic instrument 120, which is configured to analyse biological samples. Exemplarily, the first computing device 100 comprises a processor 111 (e.g. a CPU, a GPU, or the like), and a memory 112. The processor 111 is configured to carry out the method according to the first aspect of the present invention. The memory 112 may comprise a primary memory and a secondary memory (not shown). In particular, the secondary memory stores a computer program comprising instructions which, when executed by the processor 111, cause the first computing device 100 to carry out the method according to the first aspect of the present invention. The first computing device 100 may also comprise a input output (I/O) interface 110 for communicating with input/output units, such as a screen, a keyboard, a touch screen, a printer, or the like.

Furthermore, the first computing device 100 may comprise a Network Interface Controller (NIC) 114 configured to connect said device with one or more networks (e.g. an intranet, the internet, a cellular network, or the like). In other examples, the first computing device may comprise a plurality of NICs.

The first computing device 100 is in data communication 12 with the analytic instrument 120, for example by means of the NIC 114 or by other means. In particular, the first computing device 100 may be a part of the analytic instrument 120 and the data transfer may occur via a bus.

The analytic instrument 120 is configured to receive a sample container 130, such as a sample tube, containing a biological sample 131. To this aim, the analytic instrument 120 may comprise a holder 121 for holding the sample container 130, the holder comprising a recess 122 for inserting the sample container 130.

The analytic instrument 120 is also configured to draw a portion of the biological sample 131 from the sample container 130. To this aim, the analytic instrument 120 may comprise a pipetting device, such as a pipettor 123 that can be positioned in correspondence of the recess 122 along a vertical axis 140. The pipettor 123 includes a pipette 1231 for aspirating/dispensing the fluid and a motor 1232 for moving the pipette 1231 inside the sample container 130 and retracting it, in particular along the vertical axis 140.

The motor 1232 may further move the pipettor 123 along a horizontal axis 141 so that the pipettor may be positioned over other containers. In particular, the analytic instrument 120 may be equipped with at least one container 124 (called “sample vessel”) for collecting the volume drawn from the sample container 130. In one example, the sample vessel 124 may contain the whole volume extracted from the biological sample 131. In other examples, the volume may be split between the sample vessel 124 and another container 125. In particular, the container 124 may be designated for the primary volume and the container 125 may be designated for the supplementary volume(s).

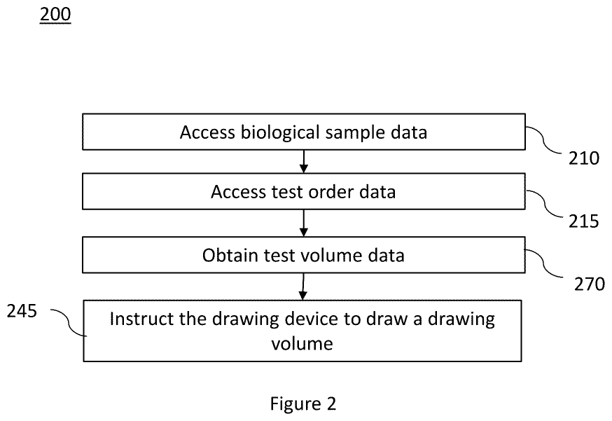

The first computing device 100 is configured to carry out the method 200 illustrated in FIG. 2. The first computing device 100 accesses test order data at step 215 and obtains test volume data at step 270, then instructs the pipetting device 123 to draw a drawing volume from the sample container 130. Optionally, the first computing device 100 may access biological sample data at step 210.

The biological sample data contain at least a value for the (initial) volume of the biological sample present in the sample container 130, i.e. before any drawing volume is aspirated from it. Furthermore, they may contain one or more flags indicating other characteristics of the sample, such as whether it has a priority and/or whether the subject is paediatric. The test order data comprises at least the first identifier, said identifier being associated with the first clinical test to be carried out on the sample.

For example, the first computing device 100 may read a barcode on the sample container 130 and retrieve the test order data and the biological sample data from a remote computer by using the information contained in the barcode. For example, the remote computer is located where the biological sample 131 was taken from the subject.

The first computing device 100 obtains test volume data by using the test order data, wherein the test volume data contain a value for the test volume, which is then used as drawing volume by the pipetting device 123. The quantity of sample indicated by the test volume always includes the quantity necessary for carrying out the first clinical test identified by the test order data (i.e. the primary volume). Furthermore, in some cases, the quantity of sample indicated by the test volume may also include the quantity necessary for carrying out the one or more supplementary tests (i.e. the supplementary volume(s)). Specifically, these are the cases in which the one or more test volume conditions are satisfied.

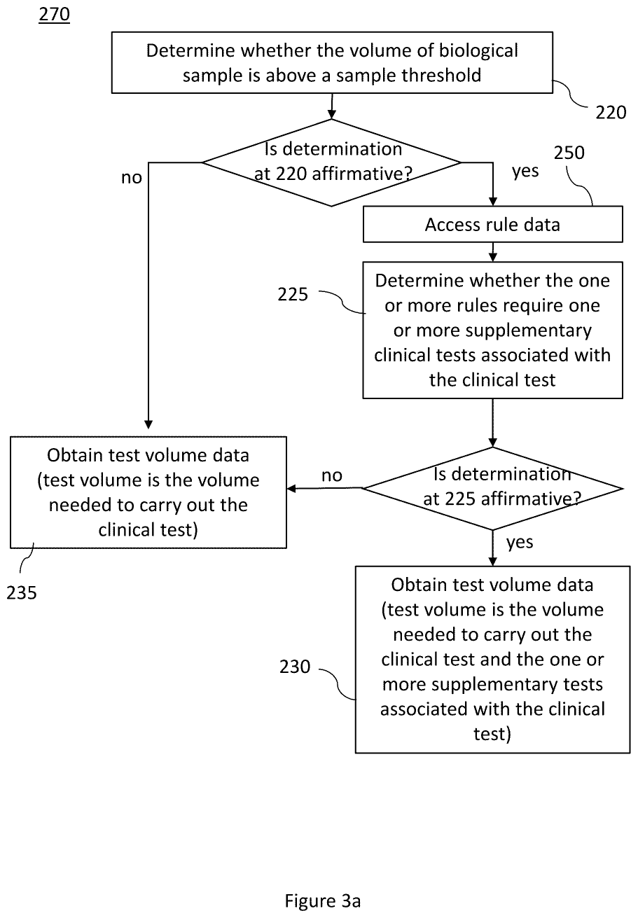

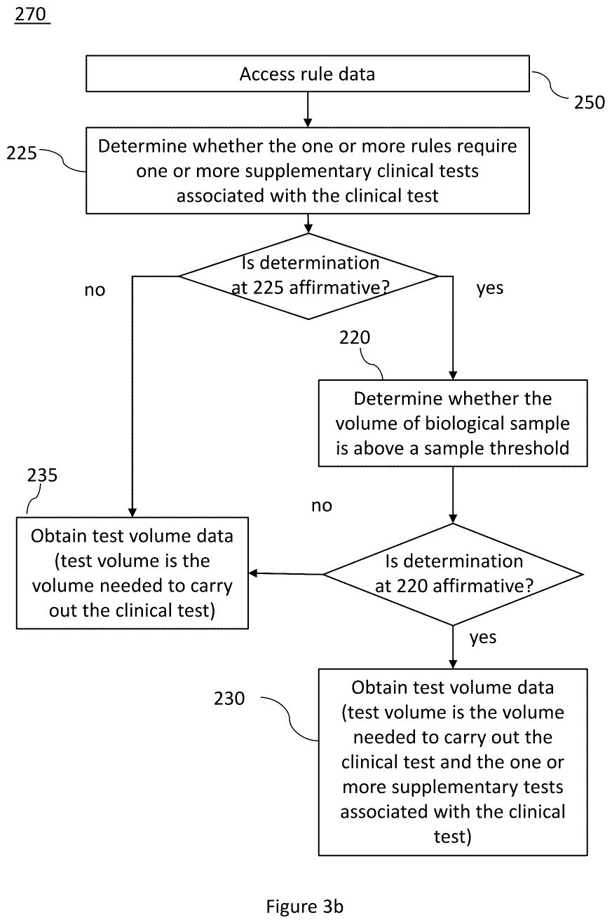

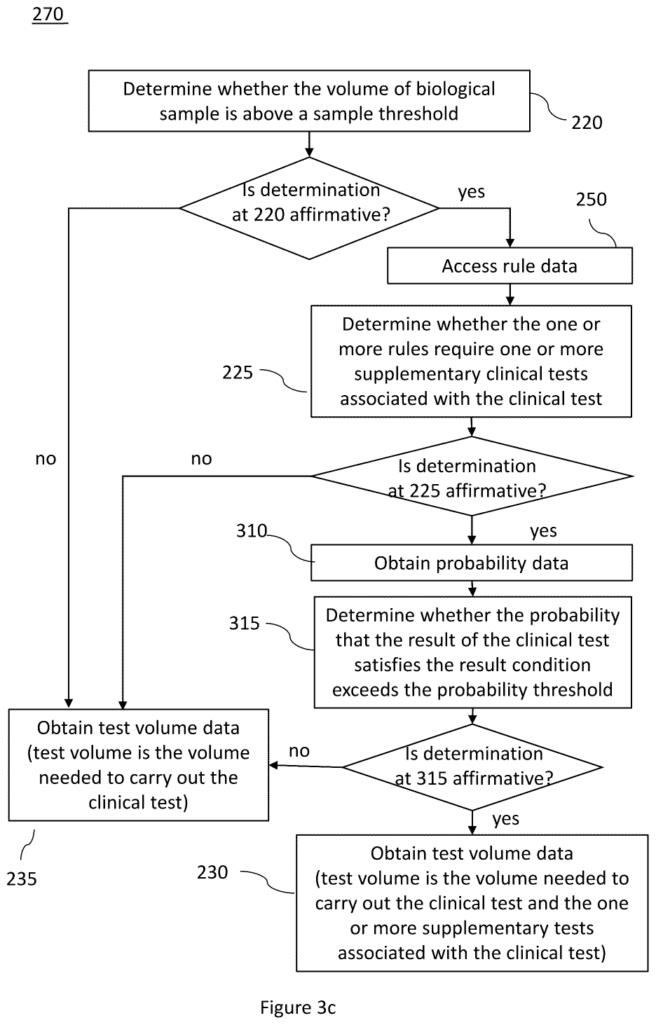

In the examples shown in FIGS. 3a-3c the first computing device 100 determines whether the one or more test volume conditions are met, in order to obtain the test volume data (step 270). A test volume condition common to the three examples is the rule condition, i.e. the condition that the one or more rules requires one or more supplementary tests to be performed whenever the supplementary test conditions are met. In particular, this rule condition is satisfied whenever at least one of the one or more rules specifies one or more supplementary tests associated with the first clinical test. In other words, the first computing device 100 checks whether there exists a possibility that one or more supplementary tests may have to be performed after the first clinical test is performed.

A supplementary test is a clinical test that is performed only when one or more respective supplementary test conditions are satisfied by the first clinical test. In particular, a supplementary test condition usually concerns the result of the first clinical test, so that it is a result condition. For example, whenever the result of the first clinical test is a numerical value exceeding a predetermined value, a supplementary test may be carried out to check whether the numerical value is correct and/or to gain an insight into why the result has such a value.

In the example of FIG. 3c, the test volume conditions further include the probability condition, i.e. the condition that the probability that the result of the first clinical test satisfies the result condition exceeds a probability threshold. In other words, the probability condition is a condition that, when met, indicates that the supplementary test will likely be carried out. The probability threshold may be a fixed value, e.g. 50%, or may be chosen on the basis of the one or more selection criteria.

The other test volume condition common to all three examples is a condition about the volume of the biological sample 131 in the sample container 130, namely that this volume exceeds a sample threshold. The sample threshold may be a fixed value for a given analytic instrument or it may be input by a user or it may be computed based on one or more input(s).

The three examples for carrying out step 270 of FIG. 2 will now be described in more detail. Some steps are common among the different examples.

In the example of FIG. 3a, the first computing device 100 determines at step 220 whether the volume of the biological sample is above a predetermined sample threshold. Whenever the volume is not greater than the sample threshold, the first computing device 100 obtains at step 235 the test volume data, wherein the test volume is equal to the volume needed to carry out the first clinical test. For instance, the first computing device 100 retrieves a value for the primary value from a database using the first identifier.

Whenever the volume in the sample container 130 is greater than the sample threshold, the first computing device 100 accesses at step 250 rule data containing one or more rules for at least some of the clinical tests of the instrument-specific set of clinical tests and, at step 225, determines whether the one or more rules specify one or more supplementary tests associated with the first clinical test.

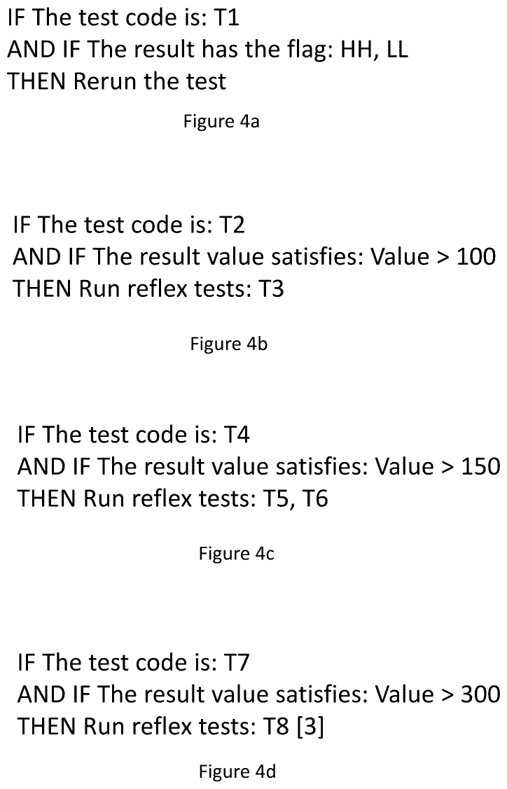

Examples of rules are shown in FIGS. 4a-4d. According to the rule shown in FIG. 4a, whenever the result of the first clinical test identified as T1 has flags “HH” and “LL”, a supplementary test should be carried out, namely T1 a second time. In other words, this rule requires a rerun of the first clinical test.

According to the rule shown in FIG. 4b, whenever the result of the first clinical test identified as T2 has a value greater than 100, a supplementary test should be carried out, namely the reflex test T3. According to the rule shown in FIG. 4c, whenever the result of the first clinical test identified as T4 has a value greater than 150, two supplementary tests should be carried out, namely the reflex tests T5 and T6. According to the rule shown in FIG. 4d, whenever the result of the first clinical test identified as T7 has a value greater than 300, the supplementary test T8 should be performed three times.

Thus, for instance, if the first clinical test is test T2 and the rule data comprise the rule of FIG. 4b, the determination at step 225 is positive. In case of an affirmative determination, the first computing device 100 obtains at step 230 as test volume the volume needed to carry out the first clinical test as well as the one or more supplementary tests. Continuing with the previous example, the test volume would be V_{T2}+V_{T3}, wherein V_{Tj} is the volume needed to carry out test Tj (j=1, 2, . . . , 8) For instance, the first computing device 100 retrieves a value for V_{T2} and a value for V_{T3} from a database using the identifiers associated with the tests T2 and T3.

If, for instance, the first clinical test is test T1, T4 or T7, respectively, and the rule data comprise the rules of FIGS. 4a to 4d, the test volume obtained at step 230 by the first computing device 100 would respectively be: 2*V_{T1}, V_{T4}+V_{T5}+V_{T6} and V_{T7}+3*V_{T8}.

If, instead, it is determined at step 225 that there is no rule concerning supplementary tests associated with the first clinical test, the test volume is obtained according to the step 235 discussed above.

In the example of FIG. 3b, the first computing device 100 performs the same steps of FIG. 3a but in a different order. Indeed, it starts with accessing the rule data (step 250) and checking the rule condition (step 225) and, then, when the determination at step 225 is affirmative, step 220 is carried out, i.e. it is determined whether the volume exceeds the sample threshold. The determination of step 220 branches off, depending on the outcome of the determination at step 220, into steps 235 and 230, namely into obtaining a test volume equal to the primary volume (step 235) or equal to a combination of the primary volume with the one or more supplementary volumes (step 230). In this example, the primary volume and the one or more supplementary volume are combined by adding them together. Step 235 also follows step 225 when the determination therein is negative.

The example of FIG. 3c includes all steps of the example of FIG. 3a and involves additional steps 310, 315. In particular, it is not sufficient that the determinations at steps 220 and 225 are positive for the test volume to be given by the combination of the primary volume with the supplementary volume(s). When the determination at step 225 is affirmative, the first computing device 100 obtains probability data at step 310, wherein the probability data comprise a probability value indicating how likely it is that the result of the first clinical test meets the result condition that would trigger the one or more supplementary tests. For example, the probability data may be generated by counting how often the carrying out of the one or more supplementary tests was triggered in past executions of the first clinical test.

Afterwards, at step 315, the first computing device 100 determines whether the probability value exceeds the probability threshold or not. In the negative case, step 235 is performed, and in the positive case, step 230 is performed. Thus, when the probability value is not greater than the probability threshold, the first computing device 100 does not include the one or more supplementary volumes in the test volume.

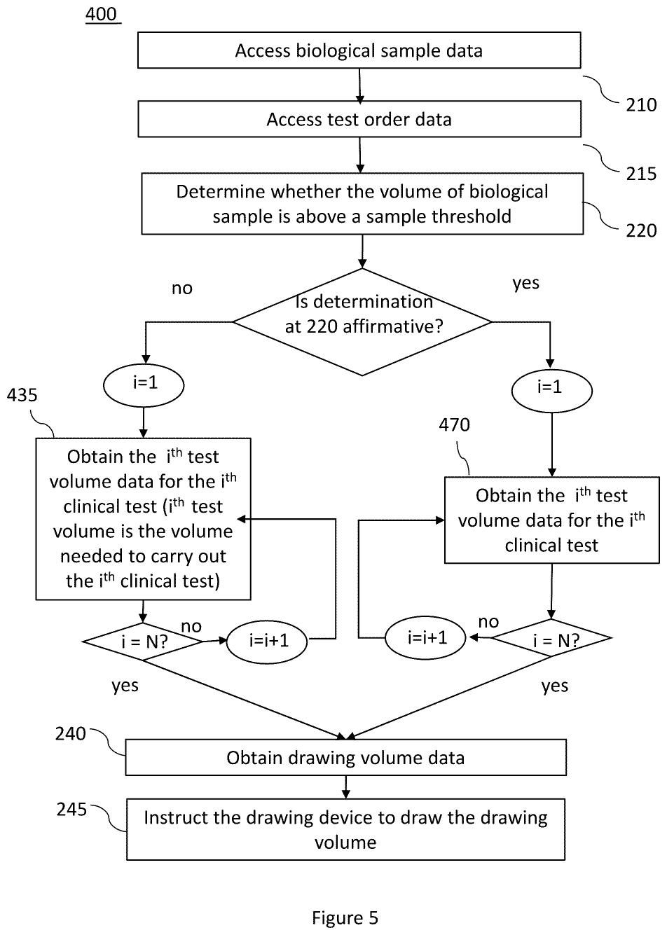

The first computing device 100 is configured to carry out the same procedure illustrated for the first clinical test also for a plurality of clinical tests specified in the test order data. This case corresponds to the method 400 shown in FIG. 5.

The first computing device 100 accesses the biological sample data at step 210, determines whether the condition on the volume within the sample container 130 is satisfied at step 220, and accesses test order data at step 215, as already explained for the previous figures. However, in this case, the test order data specify a plurality of clinical tests, N tests, e.g. by comprising a plurality of respective identifiers.

If the determination at step 220 is negative, the first computing device 100 at step 435, starting from the first clinical test of the plurality of clinical tests, obtains the first test volume data, which specify a first test volume needed to carry out the first clinical test. The first computing device 100 proceeds on doing the same for each clinical test of the plurality of tests, until it obtains the N-th test volume data specifying the N-th test volume needed to carry out the N-th test volume. Each i-th test volume (TVᵢ) is just the primary volume, i.e. the volume necessary for performing the i-th test.

If the determination at step 220 is positive, the first computing device 100 also determines an i-th test volume for each test of the plurality of clinical tests at step 470, however in this case the i-th volume may, in some cases, comprise one or more supplementary volumes, as discussed further below.

After all N test volume have been obtained, the first computing device 100 obtains drawing volume data comprising information specifying a drawing volume at step 240. Specifically, the first computing device 100 combines all the test volumes, e.g. by adding them, in order to generate the drawing volume, DV, so that DV=Σᵢ₌₁^{N}TVᵢ. Finally, as in FIG. 2, the first computing device 100 instructs, at step 245, the pipetting device 123 to draw the drawing volume from the sample container 130.

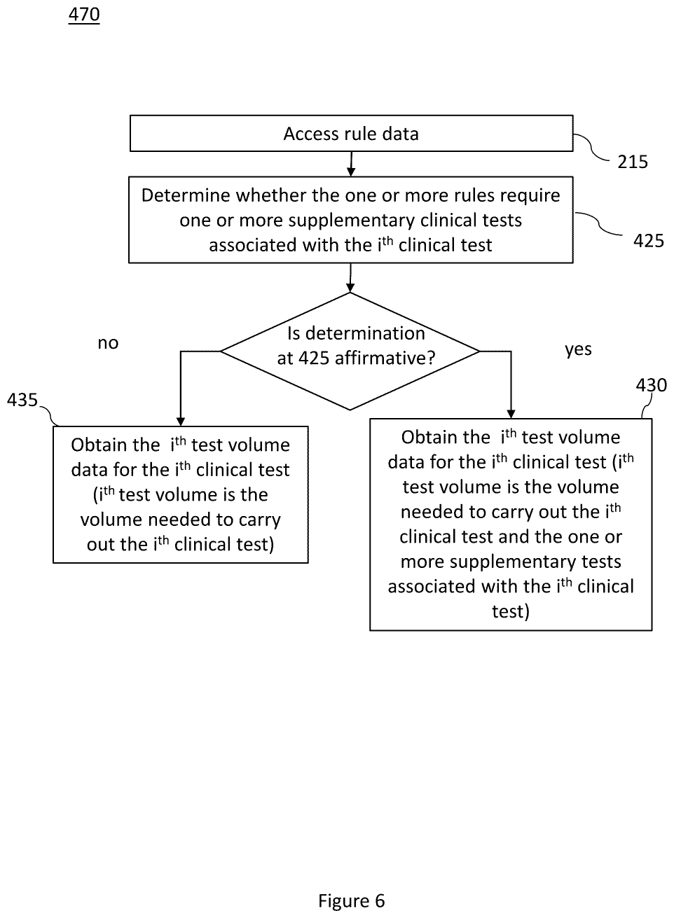

FIG. 6 shows a possible example of how each test volume is obtained for the i-th clinical test in step 470. In this example, the rule condition is applied as test volume condition. In other examples (not shown), further test volume conditions may be applied.

At step 215, the first computing device 100 access rule data and at step 425 it determines whether the one or more rules in the rule data specify one or more supplementary tests associated with the i-th clinical test. Said otherwise, the first computing device 100 determines whether the one or more rules require that one or more supplementary tests should be carried out after the i-th clinical test if one or more respective supplementary test conditions are met.

If the determination at step 425 is negative, the i-th test volume is obtained as in step 435 described with reference to FIG. 5. If the determination at step 425 is affirmative, the first computing device 100 obtains at step 430 the i-th test volume as the volume needed to carry out the i-th clinical test as well as the one or more supplementary tests associated with the i-th clinical test.

In conclusion, the first computing device 100 can control the pipetting device 123 to draw a drawing volume of sample from the sample container 130 for analysis such that an optimal balance between the efficiency of the analysis and the avoidance of sample waste is achieved.

1. A computer implemented method comprising:
accessing test order data, wherein the test order data comprise information specifying a set of clinical tests to be carried out on a biological sample, wherein the set of clinical tests comprises at least a clinical test and the biological sample is contained in a sample container;
obtaining test volume data by using the test order data, wherein the test volume data comprise information specifying a test volume; and
instructing a pipetting device of a laboratory instrument to draw a drawing volume of the biological sample from the sample container, the drawing volume being based on the test volume data;
wherein the test volume is the volume of the biological sample needed to carry out:
the clinical test, and
one or more supplementary tests associated with the clinical test, whenever one or more test volume conditions associated with the clinical test are met,
wherein the one or more test volume conditions comprise a condition that one or more rules require that the one or more supplementary tests shall be carried out whenever one or more supplementary test conditions on the clinical test are met.

2. The method of claim 1, wherein the clinical test is associated with an identifier identifying the clinical test, and wherein obtaining the test volume data comprises querying a database using the identifier.

3. The method of claim 1, wherein obtaining the test volume data comprises:
accessing rule data, the rule data comprising the one or more rules; and
determining whether the one or more test volume conditions are met.

4. The method of claim 1, wherein obtaining the test volume data comprises:
obtaining a primary value indicating a volume needed to carry out the clinical test and at least a secondary value indicating a volume needed to carry out the one or more supplementary tests associated with the clinical test; and
computing the test volume by using the primary value and the at least secondary value.

5. The method of claim 1, wherein the one or more supplementary test conditions comprise one or more result conditions on a result of the clinical test.

6. The method according to claim 5, further comprising:
obtaining, probability data, wherein the probability data comprise information specifying a probability that a result of the clinical test meets the result condition; and
determining, by using the probability data, whether the probability that the result of the clinical test satisfies the result condition exceeds a probability threshold;
wherein the one or more test volume conditions further comprise the condition that the probability that the result of the clinical test satisfies the result condition exceeds the probability threshold.

7. The method of claim 6, wherein obtaining the probability data comprises:
accessing history data, the history data comprising information specifying how often the result condition is met; and
generating the probability data by using the history data.

8. The method of claim 7, wherein the history data are collected from a plurality of laboratory instruments capable of carrying out the clinical test.

9. The method of claim 6, wherein the probability threshold is selected among a plurality of predetermined probability thresholds based on one or more selection criteria, the one or more selection criteria comprising information specifying any or any combination of: an instrument configured to carry out the clinical test, a laboratory configured to carry out the clinical test, an amount of volume of biological sample contained in the sample container, a category of a subject to whom the biological sample belongs, a priority of the biological sample.

10. The method of claim 1, wherein the one or more volume test conditions comprise a condition on an amount of volume of biological sample contained in the sample container and/or a condition on a category of a subject to whom the biological sample belongs.

11. The method of claim 1, further comprising drawing, by the pipetting device, the drawing volume of the biological sample from the sample container.

12. A computing device comprising a processor configured to perform the method of claim 1.

13. An analytic instrument configured to perform one or more clinical tests on a biological sample, wherein the at least one analytic instrument comprises:
the computing device according to claim 12; and
a pipetting device;
wherein the computing device is configured to instruct the pipetting device to draw the drawing volume of a biological sample from a sample container and the pipetting device is configured to draw the drawing volume of the biological sample from the sample container.

14. (canceled)

15. A non-transitory computer-readable storage medium comprising instructions which, when executed by a computer, cause the computer to carry out the steps of claim 1.
